(19) United States
(12) Patent Application Publication
(10) Pub. No.: US 2007/0021385 A1
(43) Pub. Date: Jan. 25, 2007

(54) Compositions and methods for wood preservation

(76) Inventors: Jun Zhang, Getzville, NY (US); Richard J. Ziobro, Peachtree City, GA (US)

Correspondence Address: HODGSON RUSS LLP, ONE M & T PLAZA, SUITE 2000, BUFFALO, NY, 14203-2391, US

(21) Appl. No.: 11/490,914

(22) Filed: Jul. 21, 2006

Related U.S. Application Data
(60) Provisional application No. 60/701,294, filed on Jul. 21, 2005.

(51) Int. Cl. A61K 31/695; A01N 43/64; A01N 65/00; A01N 55/00; A01N 43/50

(52) U.S. Cl. 514/63; 514/65; 514/397; 514/383

(57) ABSTRACT

Provided is a composition and method for the preservation of wood. The composition comprises 1) an azole and/or quaternary ammonium compound component and 2) a pyrethroid compound component such that wood treated with the composition has a greater decay resistance than wood treated to the same azole retention with the azole alone. The method comprises the application of the composition to wood.

### CROSS-REFERENCE TO RELATED APPLICATIONS

This application claims priority to U.S. Provisional application No. 60/701,294, filed on Jul. 21, 2005, the disclosure of which is hereby incorporated by reference.

### BACKGROUND

Wood and/or cellulose based products exposed in an outdoor environment are biodegradable, primarily through attack by microorganisms. As a result, they will decay, weaken in strength, and discolor. The microorganisms causing wood deterioration include brown rots such as Postia placenta, Gloeophyllum trabeum and Coniophora puteana, white rots such as Irpex lacteus and Trametes versicolor, dry rots such as Serpula lacrymans and Meruliporia incrassata and soft rots such as Cephalosporium, Acremonium and Chaetomium. Wood preservatives are well known for preserving wood and other cellulose-based materials, such as paper, particleboard, textiles, rope, etc., against organisms responsible for the deterioration of wood. Azole compounds, such as, tebuconazole, propiconazole and cyproconazole, and quaternary ammonium compounds are generally known to be effective biocides as wood preservatives. Azoles are registered as pesticides for the use in wood preservation industry, and also used in the agricultural applications to protect plants, fruits, vegetables, cereal crops and sugar corps from fungal attack. U.S. Pat. No. 5,634,967 described a wood preservative composition containing a metal compound and an azole compound. A synergistic fungicidal activity was claimed to exist between the metal compounds and azole compounds. U.S. Pat. No. 6,527,981 disclosed a fungicide system based on azoles and amine oxides. The amine oxides were found to improve the waterproofing properties and enhance the performance of azoles. U.S. Pat. No. 6,372,771 disclosed a wood preservative composition containing azole fungicides and quaternary ammonium compounds. U.S. Pat. No. 5,397,795 described a synergistic antifungal composition containing tebuconazole and propiconazole for use in wood preservation and/or protection of biodegradable materials.

Although the azole compounds are well known as fungicides, they have limited insecticidal activity. As a result, wood treated with these biocides is still subject to attack by wood-inhabiting insects, such as termites, beetles, ants, bees, wasps and so on. There has been an unmet need to produce organic based preservatives systems that will prevent wood not only from the attack by decay fungi, but also from the attack by insects. This need is solved by the subject matter disclosed herein.

### SUMMARY

Applicants have discovered that the use of pyrethroid-type insecticides as cobiocides with fungicidal azoles or quaternary ammonium compounds (quats) greatly improves the fungicidal activity of azole compounds or quaternary ammonium compounds. Examples of pyrethrins include bifenthrin, permethrin and cypermethrin.

The present invention provides compositions and methods for preservation of wood against fungal and insect attack. The composition comprises 1) an azole or quaternary ammonium-type fungicide and 2) a pyrethroid type insecticide.

Another embodiment of the present invention is a method for preserving and/or waterproofing a wood substrate by applying the composition to the wood substrate.

Provided in another embodiment of the invention is an article comprising a wood substrate to which has been applied the composition of the present invention.

Provided in yet another embodiment of the invention is a method of controlling fungi comprising applying an effective amount of the composition of the present invention to the fungi or the area on which the fungi grow.

### DETAILED DESCRIPTION

Provided herein is an organic composition and method for use thereof in treatment of cellulosic material, more particularly wood. The composition comprises an azole or quaternary ammonium fungicide compound, and a pyrethroid insecticide. The composition imparts to the treated wood resistance to both fungi and insects. Surprisingly, the fungicidal activity of azole or quaternary ammonium compounds used in combination with pyrethroid-type insecticide compounds is greater than the fungicidal activities of azoles or quaternary ammonium compounds when used alone. This is all the more unexpected in that pyrethroid insecticides, such as bifenthrin, cypermethrin, or permethrin, generally do not have fungicidal activity against brown rots or white rots. This has been confirmed by accelerated decay testing in the lab.

The compositions of the present invention have a broad spectrum of bio-efficacy against wood decay fungi, including types against which azoles and quats are known to be effective, such as, for example, brown rot fungi, white rot fungi, and soft rot fungi. Non-limiting examples of brown rot fungi include: Coniophora puteana, Serpula lacrymans, Antrodia vaillantii, Gloeophyllum trabeum, Gleoeophyllum sepiarium, Lentinum lepideus, Oligoporus placenta, Meruliporia incrassate, Daedalea quercina, Postia placenta. Non-limiting examples of white rot fungi include: Trametes versicolor, Phanerochaete chrysosporium, Pleurotus ostreatus, Schizophyllum commune, Irpex lacteus. Some non-limited examples of soft rot fungi are Chaetomium globosum, Lecythophora hoffinannii, Monodictys putredinis, Humicola alopallonella, Cephalosporium, Acremonium, and Chaetomium.

The compositions of the present invention are also effective against a broad range of insects and marine borer, including types against which pyrethroid compounds are known to be effective, such as, for example, termites, beetles, and wood-boring insects. Non-limiting examples of termites include drywood termites such as Cryptotermes and Kaloterms, and dampwood termites such as Zootermopsis, subterranean termites such as Coptotermes, Mastotermes, Reticulitermes, Schedorhinotermes, Microcerotermes, Microtermes, and Nasutitermes. Non-limiting examples of beetles include those in families such as, for example, Anoniidae, Bostrychidae, Cerambycidae, Scolytidae, Curculionidae, Lymexylonidae, and Buprestidae.

The compositions of the present invention are useful as wood preservatives for protecting wood and/or wood-based products, such as, for example, lumber, timbers, particle board, plywood, laminated veneer lumber (LVL), oriented strained board (OSB), etc. from decaying, discoloring, staining/molding, and weakening in its strength. The compositions are also useful in protecting cellulose-based products, such as textile fibers, wood pulp, wool and natural fiber, from fungi and insect attacks.

The compositions of the present invention can also be used for supplemental or remedial treatment of wood in service, such as utility poles and railroad ties. When used as remedial preservative purpose, the compositions can be in the form of a paste- or grease-type of formulations, if desired, such that the formulation has an adhesive nature and is easy to apply to a desired location. In this embodiment, the composition of the present invention can be applied to the wood surface through external coating treatment.

The present composition can also be used in combination with other known preservative and/or biocidal compounds, including copper based preservatives, such as copper-ethanolamine complexes and oxine copper; boron based preservatives, such as boric acid, sodium salts of borates; and sodium fluoride.

Fungicidal compounds which can be used in the present invention include azole compounds and quaternary ammonium compounds. Typical examples of azole compounds include: 1-[[2-(2,4-dichlorophenyl)-1,3-dioxolan-2-yl]methyl]-1H-1,2,4-triazole (azaconazole), 1-[(2RS,4RS:2RS,4SR)-4-bromo-2-(2,4-dichlorophenyl)tetrahydrofurfuryl]-1H-1,2,4-triazole (bromuconazole), (2RS,3RS;2RS,3SR)-2-(4-chlorophenyl)-3-cyclopropyl-1-(1H-1,2,4-triazol-1-yl)butan-2-ol (Cyproconazole), (2RS,3RS)-1-(2,4-dichlorophenyl)-4,4-dimethyl-2-(1H-1,2,4-triazol-1-yl)pentan-3-ol (diclobutrazol), cis-trans-3-chloro-4-[4-methyl-2-(1H-1,2,4-triazol-1-ylmethyl)-1,3-dioxolan-2-yl]phenyl 4-chlorophenyl ether (difenoconazole), (E)-(RS)-1-(2,4-dichlorophenyl)-4,4-dimethyl-2-(1H-1,2,4-triazol-1-yl)pent-1-en-3-ol (diniconazole), (E)-(R)-1-(2,4-dichlorophenyl)-4,4-dimethyl-2-(1H-1,2,4-triazol-1-yl)pent-1-en-3-ol (diniconazole-M), (2RS,3SR)-1-[3-(2-chlorophenyl)-2,3-epoxy-2-(4-fluorophenyl)propyl]-1H-1,2,4-triazole (epoxiconazole), (RS)-1-[2-(2,4-dichlorophenyl)-4-ethyl-1,3-dioxolan-2-ylmethyl]-1H-1,2,4-triazole (etaconazole), (RS)-4-(4-chlorophenyl)-2-phenyl-2-(1H-1,2,4-triazol-1-ylmethyl)butyronitrile (fenbuconazole), 3-(2,4-dichlorophenyl)-6-fluoro-2-(1H-1,2,4-triazol-1-yl)quinazolin-4(3H)-one (fluquinconazole), bis(4-fluorophenyl)(methyl)(1H-1,2,4-triazol-1-ylmethyl)silane (flusilazole), (RS)-2,4′-difluoro-α-(1H-1,2,4-triazol-1-ylmethyl)benzhydryl alcohol (flutriafol), (2RS,5RS;2RS,5SR)-5-(2,4-dichlorophenyl)tetrahydro-5-(1H-1,2,4-triazol-1-ylmethyl)-2-furyl 2,2,2-trifluoroethyl ether (furconazole), (2RS,5RS)-5-(2,4-dichlorophenyl)tetrahydro-5-(1H-1,2,4-triazol-1-ylmethyl)-2-furyl 2,2,2-trifluoroethyl ether(furconazole-cis), (RS)-2-(2,4-dichlorophenyl)-1-(1H-1,2,4-triazol-1-yl)hexan-2-ol (hexaconazole), 4-chlorobenzyl (EZ)-N-(2,4-dichlorophenyl)-2-(1H-1,2,4-triazol-1-yl)thioacetamidate (imibenconazole), (1RS,2SR,5RS;1RS,2SR,5SR)-2-(4-chlorobenzyl)-5-isopropyl-1-(1H-1,2,4-triazol-1-ylmethyl)cyclopentanol (ipconazole), (1RS,5RS;1RS,5SR)-5-(4-chlorobenzyl)-2,2-dimethyl-1-(1H-1,2,4-triazol-1-ylmethyl)cyclopentanol (metconazole), (RS)-2-(4-chlorophenyl)-2-(1H-1,2,4-triazol-1-ylmethyl)hexanenitrile (myclobutanil), (RS)-1-(2,4-dichloro-p-propylphenethyl)-1H-1,2,4-triazole(penconazole), cis-trans-1-[2-(2,4-dichlorophenyl)-4-propyl-1,3-dioxolan-2-ylmethyl]-1H-1,2,4-triazole (propiconazole), (RS)-2-[2-(1-chlorocyclopropyl)-3-(2-chlorophenyl)-2-hydroxypropyl]-2,4-dihydro-1,2,4-triazole-3-thione (prothioconazole), 3-(2,4-dichlorophenyl)-2-(1H-1,2,4-triazol-1-yl)-quinazolin-4(3H)-one (quinconazole), (RS)-2-(4-fluorophenyl)-1-(1H-1,2,4-triazol-1-yl) -3-(trimethylsilyl)propan-2-ol (simeconazole), (RS)-1-p-chlorophenyl-4,4-dimethyl-3-(1H -1,2,4-triazol-1-ylmethyl)pentan-3-ol (tebuconazole), propiconazole, (RS)-2-(2,4-dichlorophenyl)-3-(1H-1,2,4-triazol-1-yl)propyl 1,1,2,2-tetrafluoroethyl ether (tetraconazole), (RS)-1-(4-chlorophenoxy)-3,3-dimethyl-1-(1H-1,2,4-triazol-1-yl)butan-2-one (triadimefon), (1RS,2RS;1RS,2SR)-1-(4-chlorophenoxy)-3,3-dimethyl-1-(1H-1,2,4-triazol-1-yl)butan-2-ol (triadimenol), (RS)-(E)-5-(4-chlorobenzylidene)-2,2-dimethyl-1-(1H-1,2,4-triazol-1-ylmethyl)cyclopentanol (triticonazole), (E)-(RS)-1-(4-chlorophenyl)-4,4-dimethyl-2-(1H-1,2,4-triazol-1-yl)pent-1-en-3-ol (uniconazole), (E)-(s)-1-(4-chlorophenyl)-4,4-dimethyl-2-(1H-1,2,4-triazol-1-yl)pent-1-en-3-ol (uniconazole-P), and 2-(2,4-difluorophenyl)-1-(1H-1,2,4-triazole-1-yl)-3-trimethylsilyl-2-propanol. Other azole compounds include: amisulbrom, bitertanol, fluotrimazole, triazbutil, climbazole, clotrimazole, imazalil, oxpoconazole, prochloraz, triflumizole. Preferred are tebuconazole, propiconazole and cyproconazole.

Quaternary ammonium compounds which can be used in the present invention include those have the following structures: 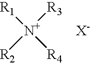
where R1, R2, R3, and R4 are independently selected from alkyl, alkenyl, alkynylor aryl groups and X⁻ selected from chloride, bromide, iodide, carbonate, bicarbonate, borate, carboxylate, hydroxide, sulfate, acetate, laurate, or other anion.

Preferred quaternary ammonium compounds include alkyldimethylbenzylammonium chloride, alkyldimethylbenzylammonium carbonate/bicarbonate, dimethyldidecylammonium chloride, dimethyldidecylammonium carbonate/bicarbonate,etc.

The pyrethroid compounds which can be used in the present invention include: acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, silafluofen. Preferred pyrethroid insecticides are bifenthrin, cypermethrin, and permethrin.

As shown in Tables 1A and 1B, when wood was treated with tebuconazole formulation alone at different retention levels expressed as kilograms per cubic meter (kg/m³), certain degree of protection against fungal attack was obtained (Table 1A), but the treated wood was subject to severe insect attack (Table 1B). When the insecticide, bifenthrin, was added to the tebuconazole formulation, the treated wood demonstrated not only great efficacy against termite attack, but also showed much greater improvement in bio-efficacy against fungal attack as shown in Table 1.

TABLE 1A
Average Decay Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated | 0.0000 | 3.8 | 0.0 | 0.0 | 0.0 |
| Wood Stakes | | | | | |
| Tebuconazole | 0.32 | 8.2 | 2.6 | 0.0 | 0.0 |
| | 0.48 | 5.8 | 0.8 | 0.0 | 0.0 |
| | 0.64 | 8.7 | 1.2 | 0.0 | 0.0 |
| Tebuconazole + | 0.32 + 0.35 | 10.0 | 10.0 | 8.8 | 8.0 |
| Bifenthrin | 0.48 + 0.35 | 10.0 | 10.0 | 10.0 | 9.8 |
| | 0.64 + 0.35 | 10.0 | 9.9 | 8.9 | 8.9 |

*The field performance test was evaluated following the procedure described in American Wood Preservers' Association (AWPA) Standard E7-01: “Standard Method of Evaluating Wood Preservatives by Field Tests with Stakes”. The rating system for decay grades are described as follows:
Decay Grades:
10 = Sound, suspicion of decay permitted
9 = Trace decay to 3% of cross section
8 = Decay from 3 to 10% of cross section
7 = Decay from 10 to 30% of cross section
6 = Decay from 30 to 50% of cross section
4 = Decay from 50 to 75% of cross section
0 = Failure due to fungal decay

TABLE 1B
Average Termite Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated | 0.0000 | 3.2 | 0.0 | 0.0 | 0.0 |
| Wood Stakes | | | | | |
| Tebuconazole | 0.32 | 4.2 | 0.6 | 0.0 | 0.0 |
| | 0.48 | 4.5 | 1.2 | 0.0 | 0.0 |
| | 0.64 | 4.8 | 2.0 | 0.0 | 0.0 |
| Tebuconazole + | 0.32 + 0.35 | 10.0 | 10.0 | 9.4 | 8.7 |
| Bifenthrin | 0.48 + 0.35 | 10.0 | 10.0 | 9.7 | 9.6 |
| | 0.64 + 0.35 | 10.0 | 10.0 | 10.0 | 8.7 |

*The field performance test was evaluated following the procedure described in American Wood Preservers' Association (AWPA) Standard E7-01: “Standard Method of Evaluating Wood Preservatives by Field Tests with Stakes”. The rating system for termite grades is described as follows:
Termite Grades:
10 = Sound, 1 to 2 small nibbles permitted
9 = Slight evidence of feeding to 3% of cross section
8 = Attack from 3 to 10% of cross section
7 = Attack from 10 to 30% of cross section
6 = Attack from 30 to 50% of cross section
4 = Attack from 50 to 75% of cross section
0 = Failure due to termite attack

The preservative compositions of the present invention can be used in the preservation of wood in a variety of ways. For example as a solution in organic solvents, an emulsion in water by emulsifying the compounds with the aid of emulsifiers, or as dispersion in water by dispersing through homogenizer or high speed agitation or through milling/grinding process or any other chemical and physical means. The fungicide and insecticide can be simultaneously or successively added to water in the presence of an emulsifier or a dispersant, followed by mixing under stirring or by grinding in a media mill. Individual concentrates of the azole or pyrethroid can be also prepared in the forms of solution, emulsion or dispersion, and then the individual concentrates of azole or pyrethroid can be mixed together and diluted to a working solution for treating wood. Non-limited examples of solvents used for dissolving azole and pyrethroid compounds include dichloromethane, hexane, toluene, alcohols such as methanol, ethanol, and 2-propanol, glycols such as ethylene glycol and propylene glycol, ethers, esters, poly-glycols, poly-ethers, amides, methylene chloride, acetone, chloroform, N,N-dimethyl octanamide, N,N-dimethyl decanamide, N-methyl 2-pyrrolidone, n-(n-octyl)-2-pyrrolidone, and combinations of the above. Typical dispersants include acrylic copolymers, aqueous solution of copolymers with pigment affinity groups, modified polyacrylate, acrylic polymer emulsions, modified lignin and the like. Emulsifiers can be anionic, cationic, or nonionic or the combinations. Examples of emulsifiers include, but are not limited to, ethyoxylated alkylphenols or amines or amides or aryl phenols or fatty esters, fatty acids and derivatives, ethoxylated alcohols and derivatives, sulfonated amine or amides and derivatives, carboxylated alcohol or alkylphenol ethoxylates and derivatives, glycol ethers or esters. Additional examples of emulsifiers can be found in McCutcheon's Emulsifiers and Detergents, 2005, the contents of which are incorporated herein by reference.

The preservative compositions of the present invention can be used in organic liquids, and such liquids can function as solvent or carrier, depending on whether the components of the present invention are solvated, or simply carried by the liquid. For example, the composition can be used in Light Organic Solvent Preservation (LOSP), where white spirits are used as the solvent/carrier. Examples of other organic solvents and/or carriers include, but are not limited to, mineral spirits, hydrocarbon solvents as described in American Wood Preservers' Association Standard P9-03, toluene, coconut oil, corn oil, soybean oil, cottonseed oil, linseed oil, peanut oil, and palm oil.

It should be noted that the use of an organic solvent or carrier can help improve the dimensional stabilization of wood, and hence reduce checking, warping or twisting. Furthermore, some organic solvents can also help improve the bio-efficacy of the preservative systems, such as by imparting a degree of water-proofing to the wood.

The fungicide and insecticide can also be dissolved in organic solvents. Non-limiting organic solvents include hydrocarbon compounds such as benzene, toluene and their derivatives, alcohols such as methanol, ethanol, ethylene glycol, propylene glycol, polyethylene glycol and their derivatives, esters such as ethyl acetate and their derivatives, ketones, dimethylsulfoxide, etc.

It should be noted that the present invention is not limited biocides dissolved in oil or water, as it is expected that particulate or micronized particulate biocides (such as, for example aqueous dispersions) will effectively preserve wood as well.

Micronized particles can be obtained by grinding the biocidal compounds using a commercially available grinding mill. Particulate compound can be wet or dry dispersed in a liquid prior to grinding. Other means of obtaining micronized particles include chemical or physical or mechanical means.

A preferred method is by grinding. One exemplary method involves the formation of a slurry comprising a dispersant, a carrier, and a powdered biocide having a particle size in the range of from 1 micron to 500 microns, and optionally, a defoamer. The slurry is transferred to a grinding mill which is prefilled with a grinding media having a size from 0.05 mm to 5 mm, and preferably between 0.1 and 1 mm. The media can be one or more of many commercially available types, including but not limited to steel shots, carbon steel shots, stannous steel shots, chrome steel shots, ceramic (for example, alumina-containing); zirconium-based, such as zirconia, zirconium silicate, zirconium oxide; stabilized zirconia such as stabilized ytz-stabilized zirconia, ceria-stabilized zirconia, stabilized magnesium oxide, stabilized aluminum oxide, etc. The medium preferably occupies 50% to 99% of the grinding chamber volume, with 75 to 95% preferred, and 80 to 90% more preferred. The bulk density of the grinding media is preferably in the range of from 0.5 kg/l to 10 kg/l, and more preferably in the range of from 2 to 5 kg/l. Agitation speed, which can vary with the size of the grinder, is generally in the range of from 1 to 5000 rpm, but can be higher or lower. Lab and commercial grinders generally run at different speeds. A set up which involves a transfer pump which repeatedly cycles the slurry between the mill and a storage tank during grinding is convenient. The transfer pump speed varies from 1 to 500 rpm, and the speeds for lab and commercial grinders can be different. During grinding, defoamer can be added if foaming is observed. During grinding, particle size distribution can be analyzed, and once particle size is within the desired specification, grinding is stopped.

The particles can be dispersed in dispersants which include standard dispersants known in the art. The dispersant can be cationic, non-ionic or anionic, and the preferred dispersants are either non-ionic or cationic. Examples of dispersants and/or surfactants which can be used in the compositions and methods of the present invention include acrylic copolymers, an aqueous solution of copolymers with pigment affinity groups, polycarboxylate ether, modified polyacrylate, acrylic polymer emulsions, modified acrylic polymers, poly carboxylic acid polymers and their salts, modified poly carboxylic acid polymers and their salts, fatty acid modified polyester, aliphatic polyether or modified aliphatic polyether, polyetherphosphate, modified maleic anhydride/styrene copolymer, lignin and the like.

For organic biocides, such as, for example, pyrethrins and azoles, the amount of dispersant is in the range of from about 1 to 200% of the weight of the biocide compounds, with a preferred range of 5 to 100%, a more preferred range of 10 to 80%, and a most preferred range of 30 to 70%.

If desired, a wetting agent can be used in the preparation of the compositions of the present invention. The amount of wetting agent is preferably in the range of from about 1 to 200% of the weight of the biocide compounds, with more preferred ranges of 5 to 100%, and 10 to 80%, and a most preferred range of 30 to 70%.

The degree of penetration and uniformity of distribution of the particles into the wood cellular structure is related to the prevalence of particles with relatively large particle size. If the biocide used in the formulation has a particle size in excess of 25 microns, the particles may be filtered by the surface of the wood and thus may not be uniformly distributed within the cell and cell wall. Furthermore, particles with long axes greater than 25 microns may clog tracheids and inhibit the uptake of additional particles. The primary entry and movement of fluids through wood tissue occurs primarily through the tracheids and border pits. Tracheids generally have a diameter of very roughly thirty microns. Fluids are transferred between wood cells by means of border pits.

The overall diameter of the border pit chambers typically varies from a several microns up to thirty microns while the diameter of the pit openings (via the microfibrils) typically varies from several hundredths of a micron to several microns.

When wood is treated with micronized preservative formulation, if the particle size of the micronized preservative is less than the diameter of the pit openings, a complete penetration and a uniform distribution of micronized preservative in wood can often take place. It should be understood that although the compositions disclosed herein contain micronized particles, they can contain particles which are not micronized, i.e., with diameters which are outside the range of from 0.001 to 25 microns.

If a particulate biocide is used, the biocide particle sizes should correspond to a distribution in which the largest particles do not appreciably inhibit wood penetration. Regardless of how many components are micronized, it is preferred that 98% (by weight) of the total number of particles in the composition have diameters which are less than 25 microns, and preferably less than 10 microns, more preferably, less than 5 micron and more preferably, less than 1 micron.

Particle size distributions which conform to the above size distribution parameters can be prepared by methods known in the art. For example, particles can be obtained by grinding a mixture of biocide and dispersant. The particle size distribution can be controlled by the ratio of dispersant to biocide, grinding times, the size of grinding media, etc. The aforementioned parameters can be adjusted in order to obtain a suitable non-clogging particle distribution.

In one embodiment particle size of the micronized particles used in the dispersion formulation disclosed herein can be micronized, i.e., with a long axis dimension between 0.001-25 microns. In a further embodiment, the particle size is between 0.001-10.0 microns. In yet another embodiment, the particle size is between 0.01 to 10.0 microns. If superior uniformity of penetration is desired, particle size of the organic biocide used in the dispersion formulation disclosed herein can be between 0.01-1.0 microns.

It is advisable to use particle size distributions which contain relatively few particle sizes outside the range of 0.001 to 25 microns. It is desirable that no more than 20 weight percent of the particles have diameters which are greater than 25 microns, and it is generally desirable that greater than 80 wt % of the particles have a diameter in the range of 0.001 to 25 microns. In more preferred embodiments, greater than 85, 90, 95 or 99 wt percent particles are in the range of 0.001 to 25 microns.

For increased certainty of complete penetration and uniformity of distribution, it is preferred that at least 50 wt % of the particles should have diameters which are less than 10 microns. More preferred are particle distributions which have at least 65 wt % of the particles with sizes of less than 10 microns. In an additional embodiment, less than 20 wt % of the particles have diameters of less than 1 micron.

The weight ratio of azole compounds, if used, to pyrethroid compounds is generally in the range of from about 1000:1 to about 0.001:1 and preferably from about 50:1 to about 0.1:1, and more preferably from 10:1 to 1:1. The weight ratio of the quaternary ammonium compounds, if used, to pyrethroid compounds is generally in the range of from about 5000:1 to about 0.01:1 and preferably from about 500:1 to about 20:1, and more preferably from about 100:1 to about 1:1

According to one embodiment of the invention, the composition can contain from about 0.5 to about 60%, preferably from about 1 to about 50%, and more preferably from about 10 to about 40% by weight of combined azole compounds or quaternary ammonium compounds and pyrethroid based upon 100% weight of total composition. The foregoing includes concentrates of the invention which can be stored or diluted as desired with a solvent or carrier and used to preserve wood. Individual concentrates of azole and/or quaternary ammonium compound or pyrethroid compounds can also be prepared and mixed together, with or without a diluent (a carrier or solvent (water, if desired) to form compositions for use in wood treatment. The above-mentioned compositions can be diluted with a desired solvent or carrier, such as water or other organic liquids, prior to use, if desired.

In general, the enhanced fungicidal effect of including pyrethroid compounds is expected over a very broad range of retentions. The wood or wood product to be preserved is preferably treated such that the azole (if used) and pyrethroid components are independently at retentions in the range of from about 0.00001 to 5 pounds per cubic foot, more preferably in the range of from about 0.0005 to about 1 pounds per cubic foot, and even more preferably in the range of from about 0.001 to about 0.1 pounds per cubic foot. The quaternary ammonium compound component, if used, is preferably in the range of from about 0.001 to 5 pounds per cubic foot, and more preferably in the range of from about 0.05 to about 1 pounds per cubic foot.

Non-biocidal additives such as fire retardants, water repellants, colorants such as pigments or dyes, emulsifying agents, dispersants, stabilizers, UV inhibitors, pigments, wax emulsions, acylate polymers, and the like may also be added to the system disclosed herein to further enhance the performance of the system or the appearance and performance of the resulting treated products. These additives may be particulate or micronized as necessary or desired.

The present invention also provides a method for preservation of wood. In one embodiment, the method comprises the steps of treating wood with a composition (treating fluid) comprising an azole or quaternary ammonium compound and a pyrethroid compound. The treating fluid may be applied to wood by impregnation, dipping, soaking, spraying, brushing, or any other means well known in the art. When used as remedial preservative purpose, the compositions can be applied to the wood surface through external coating treatment. In a preferred embodiment, vacuum and/or pressure techniques are used to impregnate the wood in accord with this invention including the standard processes, such as the “Empty Cell” process, the “Modified Full Cell” process and the “Full Cell” process, and any other vacuum and/or pressure processes which are well known to those skilled in the art.

The standard processes are defined as described in AWPA Standard C1-03 “All Timber Products—Preservative Treatment by Pressure Processes”. In the “Empty Cell” process, prior to the introduction of preservative, materials are subjected to atmospheric air pressure (Lowry) or to higher air pressures (Rueping) of the necessary intensity and duration. In the “Modified Full Cell”, prior to introduction of preservative, materials are subjected to a vacuum of less than 77 kPa (22 inch Hg) (sea level equivalent). A final vacuum of not less than 77 kPa (22 inch Hg) (sea level equivalent) shall be used. In the “Full Cell Process”, prior to introduction of preservative or during any period of condition prior to treatment, materials are subjected to a vacuum of not less than 77 kPa (22 inch Hg). A final vacuum of not less than 77 kPa (22 inch Hg) is used.

If the composition contains micronized or particulate biocides, it is preferred that the biocide be in the form of a dispersion or suspension during application to wood.

The following examples are provided to further describe certain embodiments of the invention but are in no way meant to limit the scope of the invention. Examples 1 through 13 demonstrate the formulations in the concentrated form comprising various organic biocides. Examples 14 through 22 demonstrate the preparation of treating fluids using concentrated dispersions for the treatment of wood.

### EXAMPLE 1

A 25.0% of tebuconazole concentrate was obtained by dissolving 50.0 grams of tebuconazole in 150.0 g of N-methyl-2-pyrrolidone. A 25.0% of bifenthrin concentrate was obtained by dissolving 50.0 grams of bifenthrin in 150.0 g of N-methyl-2-pyrrolidone

### EXAMPLE 2

60.0 g bifenthrin were dissolved in 125.0 g of N,N-dimethyl octanamide and 50.0 N,N-dimethyl decanamide. The solution was added to a beaker containing 200 g of water and 200 g of commercially available emulsifiers. The mixture was agitated with a high speed homogenizer for 10 minutes. A micro-emulsion containing 9.44% bifenthrin was obtained. The micro-emulsion can be mixed with water to make the work solution for treating wood samples.

### EXAMPLE 3

100.0 g of tebuconazole and 10.0 of bifenthrin were added to a beaker containing 390.0 g of N-methyl-2-pyrrolidone. The mixture was agitated for about 30 minutes, and a clear solution was obtained. The target concentration of tebuconazole and bifenthrin by weight was 20.0% and 2.0%, respectively. The resulting concentrates can be mixed with other organic solvents, such as methanol, ethanol, toluene or spirits, to make treating solutions to treat wood

### EXAMPLE 4

50.0 g of tebuconazole and 10.0 of bifenthrin were added to a beaker containing 140.0 g of N-(N-octyl)-2-pyrrolidone. The mixture was agitated for about 30 minutes, and a clear solution was obtained. The target concentration of tebuconazole and bifenthrin by weight was 25.0% and 5.0%, respectively. The resulting concentrates can be mixed with other organic solvents, such as toluene or spirits, to make treating solutions.

### EXAMPLE 5

100.0 g of propiconazole and 25.0 of bifenthrin were added to a beaker containing 1000.0 g of toluene. The mixture was agitated for about 60 minutes, and a clear solution was obtained. The target concentration of propiconazole and bifenthrin by weight was 10% and 2.5%, respectively.

### EXAMPLE 6

50.0 g of tebuconazole and 10.0 of bifenthrin were dissolved in 125.0 g of N,N-dimethyl octanamide and 50.0 N,N-dimethyl decanamide. The solution was added to a beaker containing 200 g of water and 200 g of commercially available emulsifiers. The mixture was agitated with a high speed homogenizer for 10 minutes. A micro-emulsion containing 7.87% tebuconazole and 1.57% bifenthrin was obtained. The micro-emulsion can be mixed with water indefinitely to make the work solution for treating wood samples.

### EXAMPLE 7

50.0 g of cyproconazole and 5.0 of cypermethrin were dissolved in 225.0 g of toluene. The solution was added to a beaker containing 225 g of water and 200 g of commercially available emulsifiers. The mixture was agitated with a high speed homogenizer for 10 minutes. A micro-emulsion containing 7.09% cyproconazole and 0.71% cypermethrin was obtained. The micro-emulsion can be mixed with water to make the work solution for treating wood samples.

### EXAMPLE 8

25.0 g of propiconazole, 25.0 g of tebuconazole and 25.0 of bifenthrin were dissolved in 175 g of N-(N-octyl)-2-pyrrolidone, and then 200 g of commercially available emulsifiers were added to the solution. The mixture was agitated with a high speed homogenizer for 10 minutes, and a clear solution containing 5.56% propiconazole, 5.56% tebuconazole and 5.56% bifenthrin. The resulting solution can be mixed with water to make the work solution for treating wood samples.

### EXAMPLE 9

1000 grams of tebuconazole and 200 grams of bifenthrin are mixed with a mixture of 2500 grams water and 300 grams dispersant. The mixture is mechanically mixed for about 20 minutes and then added to a grinding mill. The mixture is ground for about 120 minutes and a stable dispersion is obtained a mean particle size of 0.25 microns and 99.9% particles less than one micrometers.

### EXAMPLE 10

450 grams of tebuconazole and 45 grams of cyproconazole were mixed with a mixture of 2200 grams water and 300 grams of commercially available dispersants. The mixture is mechanically mixed for about 10 minutes and then added to a grinding mill. The mixture is ground for about 90 minutes and a stable dispersion is obtained a mean particle size of 0.22 microns and 100% particles less than one micrometers.

### EXAMPLE 11

500 grams of cyproconazole and 500 grams of permethrin are mixed with 1550 grams of water and 450 grams of dispersants. The mixture is mechanically mixed for about 15 minutes and placed in a grinding mill. The mixture is ground for about 60 minutes and a stable dispersion containing about 16.7% cyproconazole and 16.7% permethrin is obtained with a mean particle size of 0.20 micrometers.

### EXAMPLE 12

1000 grams of tebuconazole and 200 grams of bifenthrin are mixed with a mixture of 2500 grams water and 300 grams dispersant. The mixture is mechanically mixed for about 20 minutes and then added to a grinding mill. The mixture is ground for about 60 minutes and a stable dispersion is obtained with 100% particles less than one micrometers.

### EXAMPLE 13

1000 grams of tebuconazole is mixed with 2600.0 grams of water and 400.0 grams of wetting agents/dispersants. The mixture was mechanically stirred for 10 minutes. The mixture was then placed in a grinding mill and ground for about 60 minutes. A stable dispersion is obtained with a mean particle size of 0.28 microns and 100.0% particles less than one micrometer.

### EXAMPLE 14

Preservative treating solutions were prepared by the mixing the concentrates in Example 1 with ethanol. The treating solutions were used to treat wood stakes measuring 4×38×254 mm at various retentions as shown in Table 1. The treated stakes were installed in Gainesville, Fla. for field performance evaluation following the procedure described in American Wood Preservers' Association (AWPA) Standard E7-01: “Standard Method of Evaluating Wood Preservatives by Field Tests with Stakes”. Following the 48 months inspection, the results indicated that adding bifenthrin to tebuconazole not only imparted greater efficacy against termites, but also greatly improved the preservative performance against decay fungi.

### EXAMPLE 15

Preservative treating solutions were prepared by the mixing the concentrates in Example 1 with toluene. The treating solutions were used to treat wood stakes measuring 4×38×254 mm at various retentions as shown in Table 2. The treated stakes were installed in Gainesville, Fla. for field performance evaluation following the procedure described in American Wood Preservers' Association (AWPA) Standard E7-01: “Standard Method of Evaluating Wood Preservatives by Field Tests with Stakes”. Following the 48 months inspection, the results indicated that adding bifenthrin to tebuconazole not only imparted greater efficacy against termites (Table 2B), but also greatly improved the preservative performance against decay fungi (Table 2A).

TABLE 2A
Average Decay Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated Wood | 0.0000 | 3.8 | 0.0 | 0.0 | 0.0 |
| Stakes | | | | | |
| Tebuconazole | 0.32 | 8.2 | 2.6 | 0.0 | 0.0 |
| | 0.48 | 5.8 | 0.8 | 0.0 | 0.0 |
| | 0.64 | 8.7 | 1.2 | 0.0 | 0.0 |
| Tebuconazole + | 0.32 + 0.062 | 10.0 | 9.9 | 8.7 | 7.4 |
| Bifenthrin | 0.48 + 0.062 | 10.0 | 10.0 | 8.9 | 8.5 |
| | 0.64 + 0.062 | 10.0 | 10.0 | 8.9 | 8.9 |

TABLE 2B
Average Termite Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated Wood | 0.0000 | 3.2 | 0.0 | 0.0 | 0.0 |
| Stakes | | | | | |
| Tebuconazole | 0.32 | 4.2 | 0.6 | 0.0 | 0.0 |
| | 0.48 | 4.5 | 1.2 | 0.0 | 0.0 |
| | 0.64 | 4.8 | 2.0 | 0.0 | 0.0 |
| Tebuconazole + | 0.32 + 0.062 | 10.0 | 10.0 | 8.4 | 8.3 |
| Bifenthrin | 0.48 + 0.062 | 10.0 | 9.9 | 9.3 | 8.2 |
| | 0.64 + 0.062 | 10.0 | 10.0 | 9.4 | 8.6 |

### EXAMPLE 16

Preservative treating solutions were prepared by the mixing the concentrates in Example 2 and 13. The treating solutions were used to treat wood stakes measuring 4×38×254 mm at various retentions as shown in Table 3. The treated stakes were installed in Gainesville, Fla. for field performance evaluation following the procedure described in American Wood Preservers' Association (AWPA) Standard E7-01: “Standard Method of Evaluating Wood Preservatives by Field Tests with Stakes”. Following the 48 months inspection, the results indicated that adding bifenthrin to tebuconazole not only imparted greater efficacy against termites (Table 3B), but also greatly improved the preservative performance against decay fungi (Table 3A).

TABLE 3A
Average Decay Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated Wood | 0.0000 | 3.8 | 0.0 | 0.0 | 0.0 |
| Stakes | | | | | |
| Tebuconazole | 0.32 | 8.2 | 2.6 | 0.0 | 0.0 |
| | 0.48 | 5.8 | 0.8 | 0.0 | 0.0 |
| | 0.64 | 8.7 | 1.2 | 0.0 | 0.0 |
| Tebuconazole + | 0.29 + 0.056 | 9.9 | 10.0 | 9.0 | 6.9 |
| Bifenthrin | 0.45 + 0.056 | 10.0 | 10.0 | 9.9 | 9.5 |
| | 0.56 + 0.056 | 10.0 | 9.9 | 9.8 | 7.7 |

TABLE 3B
Average Termite Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| Preservative | Retention, | 12 MONTHS | 24 MONTHS | 36 MONTHS | 48 MONTHS |
| System | (kg/m³) | Decay | Decay | Decay | Decay |
| Untreated Wood | 0.0000 | 3.2 | 0.0 | 0.0 | 0.0 |
| Stakes | | | | | |
| Tebuconazole | 0.32 | 4.2 | 0.6 | 0.0 | 0.0 |
| | 0.48 | 4.5 | 1.2 | 0.0 | 0.0 |
| | 0.64 | 4.8 | 2.0 | 0.0 | 0.0 |
| Tebuconazole + | 0.29 + 0.056 | 10.0 | 9.8 | 9.1 | 9.0 |
| Bifenthrin | 0.45 + 0.056 | 10.0 | 9.9 | 9.4 | 9.4 |
| | 0.56 + 0.056 | 10.0 | 10.0 | 9.5 | 9.3 |

### EXAMPLE 17

Preservative treating solutions were prepared by mixing the concentrates in Example 2 and 10. The treating solutions were used to treat wood stakes measuring 4×38×254 mm at various retentions as shown in Table 4. In addition, preservative treating solutions were also prepared by mixing the azole concentrate in Example 10 with a non-pyrethroid insecticide, and were used to treat wood stakes. The treated stakes were installed in Gainesville, Fla. for field performance evaluation following the procedure described in American Wood Preservers' Association (AWPA) Standard E7-01: “Standard Method of Evaluating Wood Preservatives by Field Tests with Stakes”. Following the 48 months inspection, the results indicated that azole formulations containing bifenthrin demonstrated much greater decay resistance than the azole formulations containing a non-pyrethroid insecticide as shown in Table 4.

TABLE 4
Average Decay Ratings of Tebuconazole-Based Preservative Treated Wood
Stakes (4 × 38 × 254 mm) Installed in Gainesville, Florida for 48 Months*

| | | Field Exposure Time | | | |
|---|---|---|---|---|---|
| | | 12 | 24 | 36 | 48 |
| | Retention, | MONTHS | MONTHS | MONTHS | MONTHS |
| Preservative System | (kg/m³) | Decay | Decay | Decay | Decay |
| Untreated Wood Stakes | 0.0000 | 3.8 | 0.0 | 0.0 | 0.0 |
| Tebuconazole/Cyproconazole + Non- | 0.29/0.029 + 0.048 | 9.5 | 4.8 | 0.0 | 0.0 |
| pyrethroid insecticide | 0.46/0.046 + 0.048 | 9.7 | 4.5 | 0.0 | 0.0 |
| | 0.62/0.062 + 0.048 | 9.8 | 4.6 | 1.0 | 0.0 |
| Tebuconazole/ | 0.29/0.029 + 0.049 | 10.0 | 10.0 | 9.9 | 8.5 |
| Cyproconazole + | 0.46/0.046 + 0.049 | 10.0 | 9.9 | 9.9 | 9.9 |
| Bifenthrin | 0.62/0.062 + 0.049 | 10.0 | 10.0 | 9.6 | 9.6 |

### EXAMPLE 18

A preservative treating formulation is prepared by adding 0.15 kg of the cyproconazole/permethrin dispersion from Example 7 to 50.0 kg of water. This fluid is allowed to mix until a homogenous fluid is prepared. This fluid was used to treat southern pine samples measuring at 1.5″×5.5″×48″ by the full-cell process. The weight of the treated samples double and demonstrate a uniform distribution of particles throughout the wood cells and is found to be resistant to decay and insect attack.

### EXAMPLE 19

A preservative treating composition is prepared by adding 20.0 g of dispersion from Example 12 to 5.0 kg of water. The resulting fluid contains about 0.10% tebuconazole and 0.02% bifenthrin. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to contain a uniform distribution of preservative particle throughout the cross sections and is resistant to fungal and insect attack.

### EXAMPLE 20

A preservative treating composition is prepared by adding 45.0 g of dispersion from Example 8 to 5.0 kg of water. The resulting fluid contains about 0.05% tebuconazole, 0.05% propiconazole and 0.05% bifenthrin. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The treated wood is resistant to fungal and insect attack.

### EXAMPLE 21

A preservative treating composition containing 0.75% dimethyldidecylammonium carbonate/bicarbonate and 0.010% bifenthrin is prepared by mixing bifenthrin concentrate from Example 2, 50% dimethyldidecylammonium carbonate/bicarbonate and water. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to be resistant to fungal and insect attack.

### EXAMPLE 22

A preservative treating composition containing 0.50% dimethyldidecylammonium carbonate/bicarbonate and 0.009% bifenthrin is prepared by mixing bifenthrin concentrate from Example 2, 50% dimethyldidecylammonium carbonate/bicarbonate and water. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to be resistant to fungal and insect attack.

### EXAMPLE 23

Preservative treating solutions were prepared by the mixing the concentrates in Example 1 with white spirits. The treating solutions were used to treat wood stakes measuring 4×38×254 mm at various retentions as shown in Table 1. The treated stakes were installed in Gainesville, Fla. for field performance evaluation following the procedure described in American Wood Preservers' Association (AWPA) Standard E7-01: “Standard Method of Evaluating Wood Preservatives by Field Tests with Stakes”. Following the 48 months inspection, the results indicated that adding bifenthrin to tebuconazole not only imparted greater efficacy against termites, but also greatly improved the preservative performance against decay fungi.

We claim:

1) A composition for the preservation of wood comprising:
a) at least one pyrethroid compound; and
b) at least one azole compound and/or quaternary ammonium compound.

2) A composition as in claim 1 wherein the composition has a wood preservation efficacy, as determined by the American Wood Preservers' Association Standard E7-01 after a field testing time selected from the group consisting of 12, 24, 36 and 48 months, which is greater than the efficacy of the composition in the absence of the pyrethroid compounds.

3) A composition as in claim 1 wherein the composition comprises at least one azole compound and wherein the weight ratio of azole compound to pyrethroid compound is in the range of from 1000:1 to about 0.001:1.

4) A composition as in claim 1 wherein the composition comprises at least one azole compound and wherein the weight ratio of azole compound to pyrethroid compound is in the range of from 50:1 to about 0.1:1.

5) A composition as in claim 1 wherein the composition comprises at least one azole compound and wherein the weight ratio of azole compound to pyrethroid compound is in the range of from 10:1 to 1:1.

6) A composition as in claim 3 wherein one or more of the at least one azole compounds is a compound selected from the group consisting of azaconazole, bromuconazole, Cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P, 2-(2,4-difluorophenyl)-1-(1H-1,2,4-triazole-1-yl)-3-trimethylsilyl-2-propanol, amisulbrom, bitertanol, fluotrimazole, triazbutil, climbazole, clotrimazole, imazalil, oxpoconazole, prochloraz, triflumizole and combinations thereof.

7) A composition as in claim 6 wherein the at least one azole compound comprises tebuconazole, propiconazole or cyproconazole.

8) A composition as in claim 1 wherein the composition comprises at least one quaternary ammonium compound, and wherein the weight ratio of quaternary ammonium compound to pyrethroid compound is in the range of from about 5000:1 to about 0.01:1.

9) A composition as in claim 1 wherein the composition comprises at least one quaternary ammonium compound, and wherein the weight ratio of quaternary ammonium compound to pyrethroid compound is in the range of from about 500:1 to about 20:1.

10) A composition as in claim 1 wherein the composition comprises at least one quaternary ammonium compound, and wherein the weight ratio of quaternary ammonium compound to pyrethroid compound is in the range of from about 100:1 to about 1:1.

11) A composition as in claim 1 wherein the composition comprises at least one quaternary ammonium compound having the following structure: 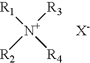
where R1, R2, R3, and R4 are independently selected from alkyl, alkenyl, alkynyl or aryl groups and X⁻ selected from chloride, bromide, iodide, carbonate, bicarbonate, borate, carboxylate, hydroxide, sulfate, acetate, or laurate.

12) A composition as in claim 11 wherein the composition comprises at least one quaternary ammonium compound selected from the group consisting of alkyldimethylbenzylammonium chloride, alkyldimethylbenzylammonium carbonate/bicarbonate, dimethyldidecylammonium chloride, and dimethyldidecylammonium carbonate/bicarbonate.

13) A composition as in claim 1 wherein the composition comprises at least one pyrethroid compound selected from the group consisting of: acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypernethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, and silafluofen.

14) A composition as in claim 13 wherein the composition comprises bifenthrin, cypermethrin, or permethrin.

15) A composition as in claim 1 wherein the composition comprises an organic carrier or solvent.

16) A composition as in claim 1 wherein the organic carrier or solvent comprises N-methyl-2-pyrrolidone, N,N-dimethyl octanamide, N,N-dimethyl decanamide, toluene, or N-(N-octyl)-2-pyrrolidone.

17) A composition as in claim 1 wherein the composition comprises an aqueous carrier or solvent.

18) A composition as in claim 1 wherein the composition comprises an emulsion in which at least one pyrethroid compound or at least one azole compound and/or quaternary ammonium compound have been dissolved in an organic solvent and emulsified in water.

19) A composition as in claim 1 wherein the composition comprises a dispersion in which at least one pyrethroid compound or at least one azole compound and/or quaternary ammonium compound have been dispersed in water.

20) A method for the preservation of wood, said method comprising the steps of:
a) applying an azole or quaternary ammonium compound to the wood;
b) applying a pyrethroid compound to the wood.

21) A method as in claim 20 wherein such that the wood preservation efficacy, as determined by the American Wood Preservers' Association Standard E7-01 after a field testing time selected from the group consisting of 12, 24, 36 and 48 months, is greater than the efficacy in the absence of the pyrethroid compound.

22) A method as in claim 20 wherein at least one azole compound is applied to the wood in step a) and wherein the weight ratio of azole compound to pyrethroid compound is in the range of from 50:1 to about 0.1:1.

23) A method as in claim 20 wherein at least one quaternary ammonium compound is applied to the wood in step a), and wherein the weight ratio of quaternary ammonium compound to pyrethroid compound is in the range of from about 5000:1 to about 0.01:1.

24) A method as in claim 20 wherein the compound in step a) and/or the compound in step b) are applied in one or more organic carriers or solvents.

25) A method as in claim 20 wherein steps a) and b) are performed simultaneously.

26) A method for the preservation of wood comprising the steps of:
a) providing a composition comprising:
1) a pyrethroid compound; and
2) an azole compound; and
b) applying the composition to wood or wood product.

27) A method as in claim 26 wherein the composition comprises an emulsion in which at least one pyrethroid compound or at least one azole compound and/or quaternary ammonium compound have been dissolved in an organic solvent and emulsified in water.

28) A method as in claim 26 wherein the composition comprises a dispersion in which at least one pyrethroid compound or at least one azole compound and/or quaternary ammonium compound have been dispersed in water.

29) A method as in claim 26 wherein the composition comprises an organic carrier or solvent.

30) A method as in claim 26 wherein the organic carrier or solvent comprises N-methyl-2-pyrrolidone, N,N-dimethyl octanamide, N,N-dimethyl decanamide, toluene, or N-(N-octyl)-2-pyrrolidone.

31) A method as in claim 26 wherein the composition comprises an aqueous carrier or solvent.

32) A method as in claim 26 wherein the composition has a wood preservation efficacy, as determined by the American Wood Preservers' Association Standard E7-01 after a field testing time selected from the group consisting of 12, 24, 36 and 48 months, is greater than the efficacy in the absence of the pyrethroid compound.

33) A method as in claim 26 wherein the composition comprises at least one azole compound and wherein the weight ratio of azole compound to pyrethroid compound in the composition is in the range of from 1000:1 to about 0.001:1.

34) A method as in claim 26 wherein the composition comprises at least one azole compound and wherein the weight ratio of azole compound to pyrethroid compound in the composition is in the range of from 50:1 to about 0.1:1.

35) A method as in claim 26 wherein the composition comprises at least one azole compound and wherein the weight ratio of azole compound to pyrethroid compound in the composition is in the range of from 10:1 to 1:1.

36) A method as in claim 26 wherein the at least one azole compound is comprises a compound selected from the group consisting of azaconazole, bromuconazole, Cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P, 2-(2,4-difluorophenyl)-1-(1H-1,2,4-triazole-1-yl)-3-trimethylsilyl-2-propanol, amisulbrom, bitertanol, fluotrimazole, triazbutil, climbazole, clotrimazole, imazalil, oxpoconazole, prochloraz and triflumizole.

37) A method as in claim 26 wherein the at least one azole compound comprises tebuconazole, propiconazole or cyproconazole.

38) A method as in claim 26 wherein the composition comprises at least one quaternary ammonium compound, and wherein the weight ratio of quaternary ammonium compound to pyrethroid compound in the composition is in the range of from about 5000:1 to about 0.01:1.

39) A method as in claim 26 wherein the composition comprises at least one quaternary ammonium compound, and wherein the weight ratio of quaternary ammonium compound to pyrethroid compound in the composition is in the range of from about 500:1 to about 20:1.

40) A method as in claim 26 wherein the composition comprises at least one quaternary ammonium compound, and wherein the weight ratio of quaternary ammonium compound to pyrethroid compound in the composition is in the range of from about 100:1 to about 1:1.

41) A method as in claim 26 wherein the composition comprises at least one pyrethroid compound selected from the group consisting of: acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, and silafluofen.

42) A method as in claim 26 wherein the composition comprises bifenthrin, cypermethrin, or permethrin.

43) A method as in claim 26 wherein the composition comprises at least one quaternary ammonium compound having the following structure: 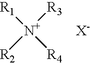
where R1, R2, R3, and R4 are independently selected from alkyl, alkenyl, alkynylor aryl groups and X⁻ selected from chloride, bromide, iodide, carbonate, bicarbonate, borate, carboxylate, hydroxide, sulfate, acetate, or laurate.

44) A method as in claim 26 wherein the composition comprises at least one quaternary ammonium compound selected from the group consisting of alkyldimethylbenzylammonium chloride, alkyldimethylbenzylammonium carbonate/bicarbonate, dimethyldidecylammonium chloride, and dimethyldidecylammonium carbonate/bicarbonate.

45) Wood preserved by the process of claim 26.
